(19) United States
(12) Patent Application Publication
(10) Pub. No.: US 2023/0162865 A1
(43) Pub. Date: May 25, 2023

(54) HEALTH MANAGEMENT SYSTEM

(71) Applicant: Nicolas Encina, Charlestown, MA (US)

(72) Inventor: Nicolas Encina, Charlestown, MA (US)

(21) Appl. No.: 17/742,918

(22) Filed: May 12, 2022

Related U.S. Application Data
(60) Provisional application No. 63/188,394, filed on May 13, 2021.

(51) Int. Cl. G16H 50/30; G16H 30/20; G16H 10/60; G16H 50/70

(52) U.S. Cl. CPC G16H 50/30; G16H 30/20; G16H 10/60; G16H 50/70

(57) ABSTRACT

A personalized healthcare management system is disclosed that includes profile storage with demographic fields for a patient, access modules for information about different conditions that have an impact on the patient's health, risk translation modules to translate the accessed information into risk indicators, and a presentation interface for the risk indicators. A statistical risk quantification engine is also disclosed that can compute risk quantification values from risk indicators and statistical models for different health conditions, and a risk aggregation engine can derive a combined health risk score from the derived values to present to the patient. A montage construction module is further disclosed that can successively access medical images from an imaging interface, and combine at least some of them into a montage to illustrate a progression of a condition, such as pregnancy, to present to the patient.

### FIELD OF THE INVENTION

This invention relates to methods and apparatus to assist in managing healthcare, including interactive systems to manage patient risk and to monitor and manage the progress of conditions such as pregnancy.

### BACKGROUND OF THE INVENTION

The field of medicine continues to steadily advance, with one estimate indicating that the body of medical knowledge doubles every 73 days. But despite this progress in knowledge, U.S. pregnant women are twice as likely to die than their mothers were 30 years ago. It therefore seems that there is a significant discrepancy between what is known about pregnancy and other conditions and how well this knowledge is used in managing these conditions.

### SUMMARY OF THE INVENTION

Several aspects of this invention are presented in this specification and its claims.

Systems according to the invention can help to improve medical care, such as care during pregnancy, by providing an interactive and organized way to evaluate and manage risk factors across a number of health determinants to prevent and treat disease. This is particularly important because many risk factors including genetic and environmental factors can have significant impact on health and healthcare spending but have tended to be overlooked in clinical care.

### BRIEF DESCRIPTION OF THE DRAWING

FIG. 1 is a block diagram of a healthcare management system according to the invention;

FIG. 2 is a diagram of a trends interface for the system of FIG. 1;

FIG. 3 is a diagram of an insights overview interface for the system of FIG. 1;

FIG. 4 is a diagram of an insights detail interface for the system of FIG. 1;

FIG. 5 is a diagram of a timeline interface for the system of FIG. 1;

FIG. 6 is a diagram of an actions interface for the system of FIG. 1;

FIG. 7 is a screenshot of an ultrasound image of a developing human fetus at approximately 34 weeks for presentation using a system such as the system of FIG. 1; and

FIG. 8 is a screenshot of a life-sized rendering of a fetus in a virtual 3-Dimensional and dynamic display.

### DETAILED DESCRIPTION OF AN ILLUSTRATIVE EMBODIMENT

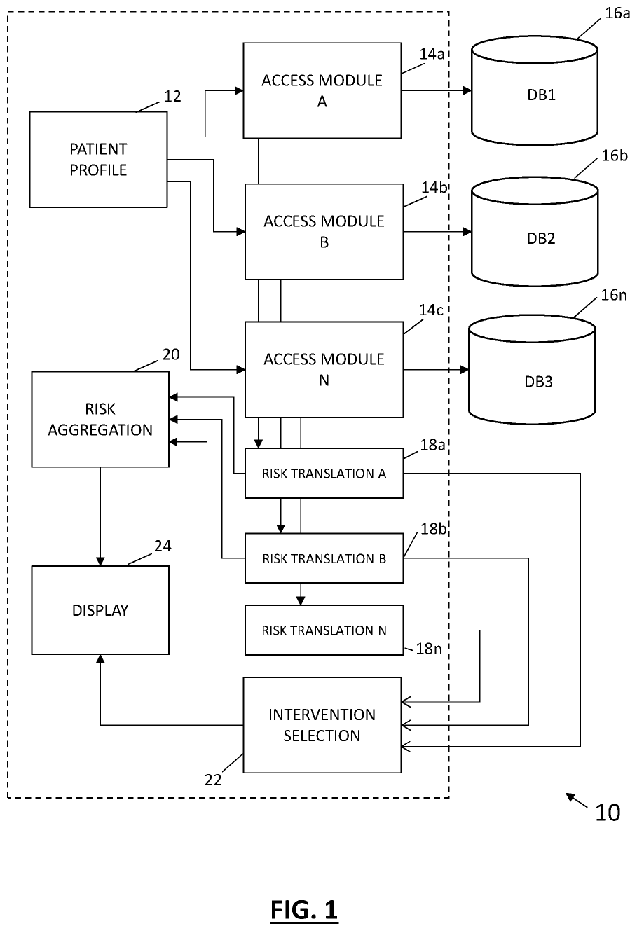

Referring to FIG. 1, a healthcare management system according to the invention, such as a pregnancy management system 10, can include a patient profile module for receiving and storing information about the patient. This information can include any information that is relevant to the patient's care, such as demographic information about the patient, information about the patient's medical conditions, and information about the patient's life conditions.

The system 10 can also include a number of information access modules 14a, 14b, . . 14n that are responsive to the patient profile module 12 and each allow the system to access information about the patient from one or more of a number of different data sources 16a, 16b, . . 16n. These data sources can include in-house or third-party sources of data relating to the patient's health, such as sources of clinical data (e.g., data about infections and fetal stress) and laboratory data (e.g. genetic and hormonal data). They can also include sources of data about life conditions, such as sources of socioeconomic data (e.g. data about ethnicity and crime), environmental data, (e.g. data about exposure to lead and other pollutants) and behavioral data (e.g. data about nutrition and substance use). Some illustrative data sources include the U.S. Census Bureau, Centers for Disease Control (CDC), the Federal Bureau of Investigation (FBI), and the Environmental Protection Agency (EPA). Data can also be obtained from a variety of other sources, such as from medical records and insurance claims as well as from wearable devices.

The system 10 can further include a number of risk translation modules 18a, 18b, . . 18n that can each be responsive to one or more of the information access modules 14a, 14b, . . 14n to derive individual risk scores from information retrieved by the information access modules. A risk aggregation module 20 is responsive to the risk translation modules to derive an aggregated risk score from the individual risk scores for the patient. An intervention selection module 22 is also responsive to the risk translation modules to derive suggested interventions. A display module 24 is responsive to the risk aggregation module and the intervention selection module to display results from those modules.

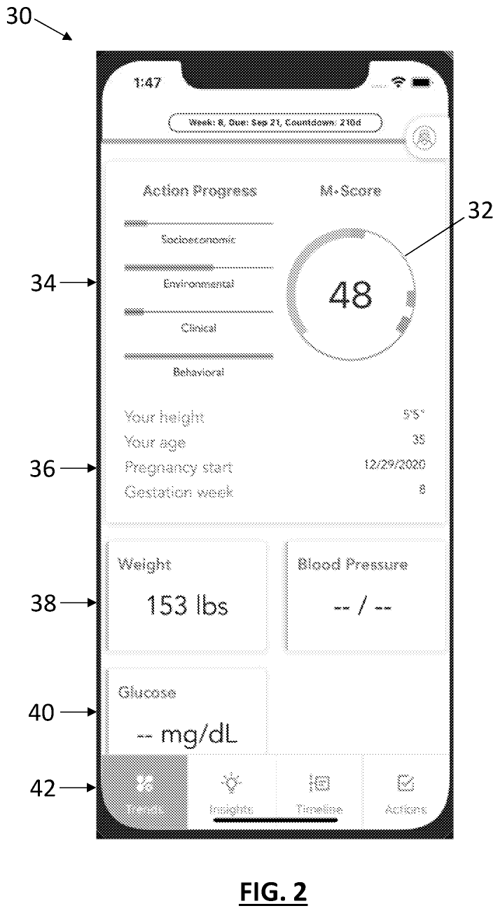

The system can be operated by a caregiver and/or patient using a standard computer platform, such as a workstation, laptop, or smart phone. Referring to FIGS. 2-6, in one embodiment a patient focused pregnancy management system uses a smart phone in communication with a server to provide a series of patient interfaces including a trends interface 30, an insights overview interface 50, an insights detail interface 60, a timeline interface 70, and an actions interface 90 that can each be selected using an interface selection control 42.

Referring to FIG. 2, the trends interface 30 can present a risk reporting area 34 that presents individual and aggregated risks. In this embodiment, the individual scores are shown using a colored bar graph metaphor. The aggregated risk score is presented inside a compound circular device 32 that also employs a segmented, axial colored edge to show how the individual risk scores combine to produce the aggregated risk score. This presentation method is presently preferred but other interface metaphors could also be used in the risk reporting area.

The trends interface 30 can also include profile areas 36, 38, 40 that show profile information for the user. These areas can show patient information, such as demographic information 36, weight and blood pressure information 38, and glucose readings 40. This information can be populated directly through the smart phone and/or derived from other systems.

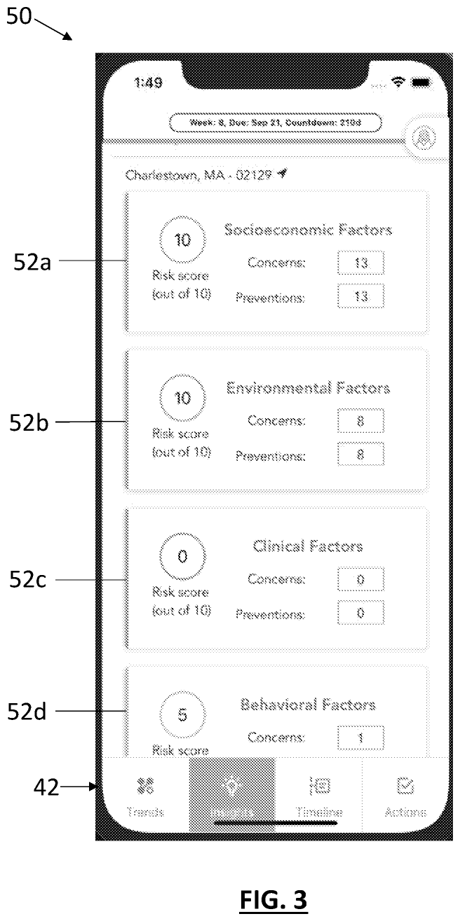

Referring to FIG. 3, the insights overview interface 50 can include a variety of risk profile areas 52a, 52b, 52c, 52d . . . These risk profile areas each show a score for a different type of risk. They can also show concern and prevention counts. Each of these areas can correspond to one of several insights detail interfaces, which can be reached by actuating that risk profile area.

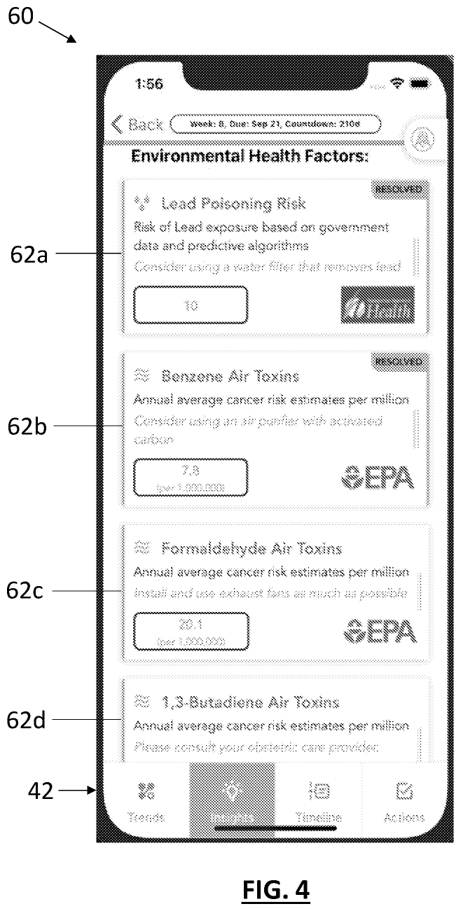

Referring to FIG. 4, each insights detail interface (e.g., 60) can show more detailed information about risks, such as environmental risks, for one of the risk profile areas in the insights overview interface. In one embodiment, the insights detail interface includes a number of risk detail areas 62a, 62b, 62c, 62d that each present information about one particular environmental risk, such as lead poisoning risk, benzene air toxin risk, formaldehyde air toxin risk, and 1,3 butadiene air toxin risk. Some more illustrative examples of risk areas are presented in table 1.

TABLE 1
Illustrative risk factors

| Behavioral | Environmental | Clinical | Laboratory | Socioeconomic |
|---|---|---|---|---|
| Smoking | Lead exposure | Intrauterine | Genetic | Ethnicity |
| Alcohol | Tobacco smoke | infections | Inflammation | Maternal stress |
| Recreational | Sulfur dioxide | Uteroplacental | (interleukins) | (life events- |
| drugs (cocaine) | and other | thrombosis | Hormonal | divorce, job |
| poor nutrition | pollutants | Fetal stress | (GGH) | loss) |
| (poor iron, long | | | Stress (cortisol) | Domestic |
| chain fats, folate) | | | Vaginal (lactic, | violence |
| | | | TIMP) | Neighborhood |
| | | | Placental (PIGF, | crime |
| | | | fibronectin) | |

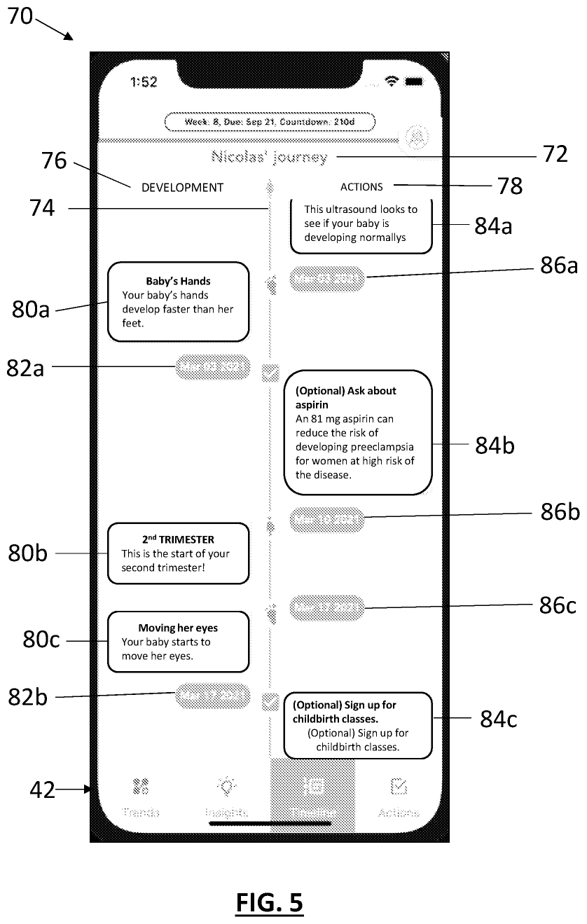

Referring to FIG. 5, the timeline interface 70 organizes items on a timeline to allow a patient to follow the progress of a treatment plan or condition, such as pregnancy. In one embodiment, the timeline view includes a personalized caption 72, a development column 76, and an actions column 78. The development column can include a succession of development milestone items 80a, 80b, 80c . . . that each present a milestone such as a fetal developmental milestone. The actions column can include a succession of recommended and/or optional action items 80a, 80b, 80c . . . for the patient to consider. Date items 82a, 82b . . . and 86a, 86b, 86c . . . can separate and organize the items in the two columns.

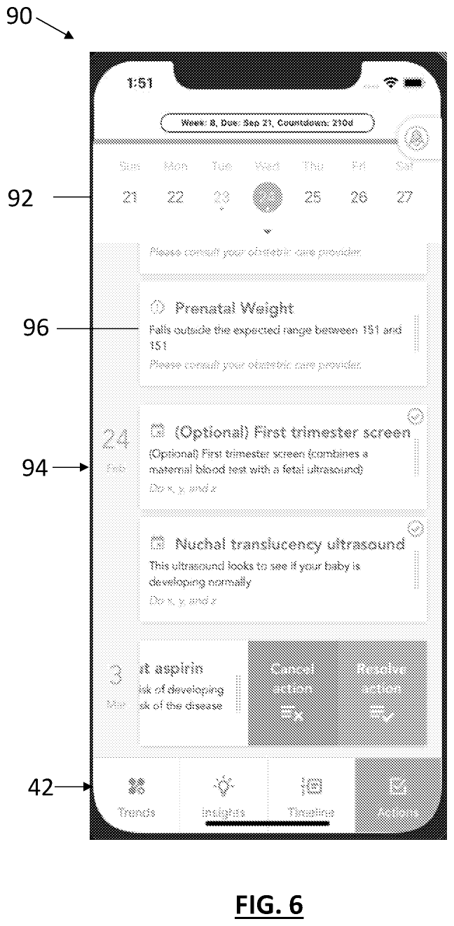

Referring to FIG. 6, the actions interface 90 can include a date control 92 to allow the patient to navigate through a series 96 of the recommended and/or optional action items. The different interfaces presented by the system are presently contemplated as well suited to the healthcare management system 10, but other approaches to the user interface could also be employed.

Overall, the system can provide a trusted platform to help to inform the patient about what they can do to understand, prepare for, and/or avoid potential complications, such as preterm birth, gestational diabetes, preeclampsia, caesarean section, embolism, hemorrhage, infection, and cardiomyopathy. The system can also provide peace of mind, help to interest, engage, and empower the patient in his or her own care, and promote better patient compliance with courses of treatment. It may further help to standardize and coordinate care, as well as to reduce unnecessary procedures and expenses.

These objectives are aided by the calculation of the aggregated risk score, which is a multi-factorial weighted mathematical construct to assess an overall patient status that accounts for their “assets and liabilities” in health (see FIG. 2). After analyzing the incoming data, the user is credited for factors working in their favor (e.g., clean air, high median home income, etc.) and charged for at-risk factors. The goal is to drive each user to 100% by giving them actions to address the difference between their score and 100%. These recommendations can take on various forms, from evidence-based clinical guidelines, to common-sense recommendations (e.g., if Radium is detected in the local water system, simply don't drink the tap water), to alerting the user of risks (as a bare minimum). If we refer to evidence that three out of five maternal deaths are attributed to errors in diagnosis, the system can potentially save countless lives, or avoid substantial pain and suffering, by simply alerting mother and clinician that there is a statistical risk of harm.

Risks are categorized by determinant (see FIG. 3) in order to further educate the user on the concepts of precision medicine, and further listing the actual risks factors (see FIG. 4) in context of authoritative sources for further reading. Once the aggregated risk score has been calculated, the system can further ‘gamify’ the experience by assigning points to every actionable intervention such that the user may engage with the system, anticipate actions and preventions via a timeline (see FIG. 5), perform the relevant tasks, complete them on the phone (see FIG. 6), and get credit which translates into a higher aggregated score (see FIG. 1). By closing this loop the system can effectively create a virtuous gamification cycle of: Educate. . . Inform. . . Engage. . . Act. . . Credit, which repeats itself through the user's experience with the system.

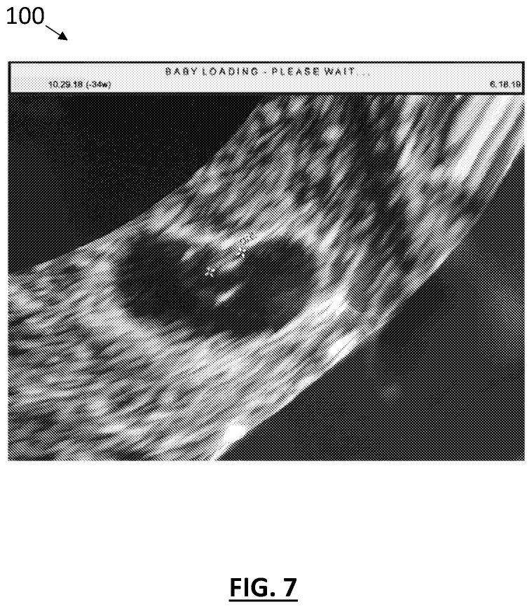

Referring to FIG. 7, the system 10 can also include a development montage creation module that provides a development montage interface 100 to show the progression of a condition, such as the development of a fetus during pregnancy. This type of interface preferably presents a montage of separate images from the fetus during the course of a pregnancy. These images can be obtained from different imaging sessions, such as ultrasound imaging sessions, as the pregnancy progresses. They can be obtained directly from the ultrasound imaging instrument, or by entering them later, such as using a camera interface on the smart phone to acquire an image from a display screen on the ultrasound imaging instrument or a paper printout from the ultrasound imaging instrument. The montage is preferably updated on an ongoing basis during the pregnancy and can be formatted in any suitable way, such as using the Graphics Interchange Format (GIF).

The system can also receive audio information such as from a fetal Doppler ultrasound heartbeat monitor and overlay this information as a soundtrack on the montage. This audio information can be received directly from the instrument or it can be acquired indirectly, such as using the microphone of the smart phone. The system 10 can employ pattern recognition to recognize and align features of the fetus in the images and match audio features with image features. The system can also employ machine learning to stitch” or “morph” the images together such that the montage actually looks like a real-time rendering of the growing baby. It may also be possible to apply analytics to these images in order to (1) offer diagnostic support and/or (2) project what the baby may look like once born. The montage creation module can also add intermediate views, a generic heartbeat soundtrack if one is missing, or make other enhancements to the montage. The montage creation module can also be provided as a standalone application separate from the rest of the healthcare management system 10.

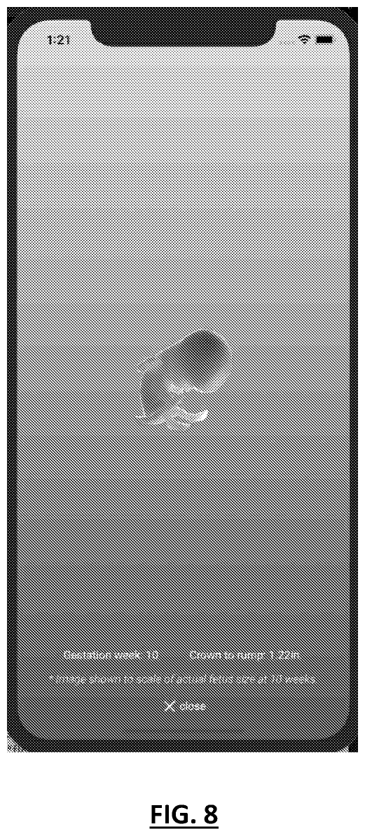

Referring to FIG. 8, the system 10 can also include a life-sized rendering of the fetus in a virtual 3-Dimensional and dynamic display. This type of interface would present the user with a life-like view of their unborn fetus at the particular time of use. Average fetus measurements were obtained by week of pregnancy and converted to device-independent-pixels in order to accurately represent physical measurements (mm, cm, inches) on any digital device. Graphical renderings of fetuses at each week of pregnancy were gathered and isolated from their backgrounds so that they may be superimposed over a stationary background that is independent of the fetus image. These time-dependent fetus illustrations are presented to the user in accordance to their week of pregnancy at time of viewing. The device's gyroscope is then used to capture the device motion so that movement in the X/Y/Z coordinates translate into perceived movement by the fetus as it “floats” above a stationary background. The user is then given various options for further inspection, either manually through hand-gestures and finger movements, or with presented statistics and relevant materials and representations.

Referring to Appendix 1, another version of a healthcare management system according to the invention is presented in a screenshot and description format. This version includes many of the features described above but uses a different user interface metaphors and includes some additional features. Some of the additional features include adding notes and/or changing the due date, an agenda page that categorizes events, actions and completed, kick tracking, a printout feature that allows the user to take a printout to a doctor's appointment, and a weight tracking feature.

The system described above has been implemented with a server running special-purpose software programs on a general-purpose computer platform, such as a Microsoft Windows or UNIX/Linux-based platform, and communicating with patient and/or caregiver smart phones, such as Android or iOS-Based smart phones. But they can also be implemented in whole or in part with other platforms or in other ways such as using dedicated hardware and/or in cloud-based or virtualized environments. And while the system can be broken into the series of modules and steps shown for illustration purposes, one of ordinary skill in the art would recognize that it is also possible to combine them and/or split them differently to achieve a different breakdown, and that the functions of such modules and steps can be arbitrarily distributed and intermingled within different entities, such as routines, files, and/or machines. Moreover, different providers can develop and operate different parts of the system.

The present invention has now been described in connection with a number of specific embodiments thereof. However, numerous modifications which are contemplated as falling within the scope of the present invention should now be apparent to those skilled in the art. Therefore, it is intended that the scope of the present invention be limited only by the scope of the claims appended hereto. In addition, the order of presentation of the claims should not be construed to limit the scope of any particular term in the claims.

1-24. (canceled).

25. A personalized healthcare management system, comprising:
profile storage for a patient profile that includes one or more demographic fields for the patient,
a plurality of access modules each responsive to the profile storage and operative to access information about different life conditions that have an impact on a patient's health,
a plurality of risk translation modules each operative to translate the accessed information attribute for the patient's life conditions into a risk indicator, and
a presentation interface operative to present the risk indicators to the patient.

26. The system of claim 24 wherein at least some of the access modules are responsive to public databases.

27. The system of claim 24 wherein at least some of the access modules use zip codes to access the patient attribute information.

28. The system of claim 24 wherein at least some of the access modules are constructed to access socioeconomic, behavioral, and environmental data for the patient's residential situation.

29. The system of claim 24 further including a statistical risk quantification engine responsive to the risk indicators and to statistical models for a each of a plurality of different health conditions and operative to compute risk quantification from the risk indicators for at least some of the modeled health conditions,
a risk aggregation engine responsive to the risk qualification engine to derive a combined health risk score from a plurality of derived quantification values from the statistical risk quantification engine, and
a combined risk score presentation interface responsive to the risk aggregation engine and operative to present the combined health risk score to the patient.

30. The system of claim 5 further including intervention retrieval and presentation logic responsive to the risk indicators and operative to present suggested interventions that the patient can make to improve the combined health risk score.

31. The system of claim 6 further including a timeline presentation interface operative to present the suggested interventions on a timeline.

32. The system of claim 5 wherein the combined risk score presentation interface is operative to present the combined health risk score in connection with the underlying types of risk that drive it.

33. The system of claim 5 further including a risk presentation interface responsive to the risk qualification engine to present information about the individual risk quantification values to the patient.

34. The system of claim 24 further including a notes collection interface.

35. The system of claim 24 further including a control to allow for changing a pregnancy due date.

36. The system of claim 24 further including an agenda page that categorizes events, actions and completed.

37. The system of claim 24 further including a kick tracking interface.

38. The system of claim 24 further including a printout feature that allows the user to take a printout to a doctor's appointment.

39. The system of claim 24 further including and a weight tracking interface that allows the user to track weight gain during pregnancy and put this weight gain in context relative to expected ranges.

40. A personalized healthcare management system, comprising:
a source of risk indicators for a patient,
a statistical risk quantification engine responsive to the risk indicators and to statistical models for a each of a plurality of different health conditions and operative to compute risk quantification values from the risk indicators for at least some of the modeled health conditions,
a risk aggregation engine responsive to the risk qualification engine to derive a combined health risk score from a plurality of derived quantification values from the statistical risk quantification engine, and
a combined risk score presentation interface responsive to the risk aggregation engine and operative to present the combined health risk score to the patient.

41. A personalized healthcare management system, comprising:
a digital imaging interface operative to receive and store a series of successive digital medical images of a patient,
a montage construction module responsive to the digital imaging interface to successively access the received medical images from the imaging interface and to combine at least some of the images into a montage to illustrate a progression of a condition in the patient, and
a presentation interface operative to present the montage to the patient.

42. The system of claim 41 further including a digital audio interface operative to receive a series of sounds from the patient and wherein the montage construction module is responsive to the digital audio interface to combine audio information from the digital audio interface with the images in the montage.

43. The system of claim 42 wherein the digital imaging interface is operative to receive a series of prenatal ultrasound images during a pregnancy of the patient.

44. The system of claim 41 wherein the digital imaging interface is operative to receive a series of prenatal ultrasound images during a pregnancy of the patient.

45. The system of claim 41 wherein the montage construction module is operative to update the montage on an ongoing basis as the medical images are received from separate imaging sessions.

46. The system of claim 41 wherein the imaging interface includes a smart phone camera and the montage construction module is an application running on the smart phone.

47. The system of claim 41 wherein the montage construction module employs results of machine learning applied to successions of images from other patients to construct the montages.

48. The system of claim 41 wherein the montage construction module includes pattern recognition to match features in images from other patients.

49. The system of claim 41 wherein the montage construction module is operative to construct the montage as a video sequence that includes at least portions of some of the received digital medical images.

50. A personalized healthcare management system, comprising:
a pregnancy stage input operative to receive an indication the stage of user's pregnancy, and
a fetal imaging construction module operative to present a constructed spatial image of the fetus at the stage received by the pregnancy stage input.

51. The system of claim 50 wherein the fetal image construction module includes float simulation logic operative to present the constructed spatial image of the fetus as a floating fetus.

52. The system of claim 50 wherein the fetal image construction module includes device-independent pixel adjustment logic operative to accurately represent physical measurement on different types of digital devices.

53. The system of claim 52 wherein the fetal image construction module includes zoom logic to allow a user to adjust the size of the constructed image.

54. The system of claim 50 wherein the fetal image construction module includes curvature adjustment logic to adjust an overall curvature of the representation of the fetus.
